(19) United States
(12) Patent Application Publication
(10) Pub. No.: US 2021/0308336 A1
(43) Pub. Date: Oct. 7, 2021

(54) ADIPOSE TISSUE MATRIX WITH TROPOELASTIN

(71) Applicant: LifeCell Corporation, Madison, NJ (US)

(72) Inventors: Hui Xu, Plainsboro, NJ (US); Alexandra Pastino, Cherry Hill, NJ (US); Carrie Fang, Madison, NJ (US)

(21) Appl. No.: 17/221,207

(22) Filed: Apr. 2, 2021

Related U.S. Application Data
(60) Provisional application No. 63/004,794, filed on Apr. 3, 2020.

(51) Int. Cl. A61L 27/56; A61L 27/36; A61L 27/22; A61F 2/12; B29C 67/20

(52) U.S. Cl. CPC A61L 27/56; A61L 27/3633; A61L 27/227; A61L 27/3687; B29L 2031/7532; A61F 2/12; B29C 67/20; A61L 2430/40; A61L 2430/04; A61L 27/3691

(57) ABSTRACT

The present disclosure provides tissue products produced from adipose tissues, as well as methods for producing such tissue products. The tissue products can include acellular tissue matrices for treatment of a breast or particulate products. The products can include adipose matrix and tropoelastin.

This application claims priority under 35 USC § 119 to U.S. Provisional Application No. 63/004,794, which was filed on Apr. 3, 2020, and is incorporated by reference in its entirety.

### REFERENCE TO SEQUENCE LISTING

The application contains a Sequence Listing in computer readable form, which has been submitted electronically via EFS-web in ASCII format. Said ASCII copy, created on Jun. 7, 2021, is named 128196-26902_Seq_Listing is 6,840 bytes in size. The computer readable form of the sequence listing is part of the specification or is otherwise incorporated herein by reference.

The present disclosure relates to tissue products, and more particularly, to extracellular tissue matrices made from adipose tissue and including tropoelastin.

Various tissue-derived products are used to regenerate, repair, or otherwise treat diseased or damaged tissues and organs. Such products can include tissue grafts and/or processed tissues (e.g., acellular tissue matrices from skin, intestine, or other tissues, with or without cell seeding). Such products generally have properties determined by the tissue source (i.e., tissue type and animal from which it originated) and the processing parameters used to produce the tissue products. Since tissue products are often used for surgical applications and/or tissue replacement or augmentation, the products should support tissue growth and regeneration, as desired for the selected implantation site. The present disclosure provides adipose tissue products that can allow improved tissue growth and regeneration for various applications, such as breast implants. The tissue products can further include tropoelastin to improve biologic or mechanical properties. The products can be cross-linked to maintain a desired shape when implanted.

According to certain embodiments, methods for producing tissue products are provided. The methods can include selecting an adipose tissue; mechanically processing the adipose tissue to reduce the tissue size; treating the mechanically processed tissue to remove substantially all cellular material from the tissue; suspending the tissue in a liquid to form a suspension; and drying the suspension in a mold to form a porous sponge. In some embodiments, the suspension is dried and subject to treatment such as dehydrothermal cross-linking. The tissue product can include tropoelastin.

In various embodiments, the adipose tissue is processed to control certain mechanical properties. For example, the processed tissue can be cross-linked to produce a stable three-dimensional structure. Additionally, or alternatively, the percent solid content of the sponge or suspension can be controlled, as discussed in further detail below. Furthermore, in some embodiments, tropoelastin may be added to the composition before forming a suspension.

Also provided herein are tissue products made by the disclosed processes.

In some embodiments, the tissue products include a decellularized adipose extracellular tissue matrix, wherein the tissue matrix has been formed into a predetermined three-dimensional shape, and wherein the tissue matrix is partially cross-linked to maintain the three-dimensional shape. The products may include a desired amount of tropoelastin.

Also provided herein is a tissue product comprising a breast implant. The implant can comprise an adipose tissue matrix formed with a desired set of mechanical properties controlled by cross-linking and/or percent solids. The product can further include tropoelastin.

Also provided herein is a tissue product comprising an injectable or particulate adipose matrix. The implant can be at least partially cross-linked solids. The product can further include tropoelastin.

Further provided herein are methods of treatment comprising the steps of selecting a tissue site and implanting the tissue products disclosed herein into the tissue site.

### BRIEF DESCRIPTION OF THE DRAWINGS

FIG. 1 is a flow chart outlining a process for producing an adipose tissue matrix sponge, according to certain embodiments.

FIG. 2 is a side view of a biologic breast implant, according to certain embodiments.

FIG. 3A is a perspective view of a configuration for a breast implant, according to certain embodiments.

FIG. 3B is a perspective view of another configuration for a breast implant, according to certain embodiments.

FIG. 3C is a perspective view of another configuration for a breast implant, according to certain embodiments.

FIG. 4 illustrates implantation of a system for surgical breast procedures, according to certain embodiments.

FIGS. 5A-5G are histologic images showing the effect of EDC crosslinking on adipogenesis.

FIG. 6A is a bar graph showing the effect of adipose matrix solid content on compressive strength.

FIG. 6B is a bar graph showing the effect of adipose matrix solid content on recovery percentage.

FIG. 6C is a bar graph showing the effect of adipose matrix solid content on elasticity.

FIG. 6D is a bar graph showing the effect of adipose matrix solid content on modulus.

FIG. 7A is a bar graph showing the effect of varying crosslinker content or tropoelastin content on compressive strength of an adipose matrix product.

FIG. 7B is a bar graph showing the effect of varying crosslinker content or tropoelastin content on recovery percentage of an adipose matrix.

FIG. 7C is a bar graph showing the effect of varying crosslinker content or tropoelastin content on modulus of an adipose matrix product

FIGS. 8A-8D are representative histological images showing the effect of tropoelastin addition on adipogenesis with or without EDC crosslinking the adipose matrix product in a rat model at 8-weeks after implantation.

FIG. 9 is a graph of adipose growth for adipose matrix with or without EDC crosslinking and with or without added tropoelastin 8-weeks after implantation.

FIGS. 10A-10D are representative histological images showing the effect of tropoelastin addition on adipogenesis with or without EDC crosslinking the adipose matrix product in a rat model at 16-weeks after implantation.

FIG. 11 is a graph of adipose growth for adipose matrix with or without EDC crosslinking and with or without added tropoelastin 16-weeks after implantation.

FIGS. 12A-12B are Mason's trichrome stained sections of explants at 16 weeks showing persistence of TE.

### DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

As used herein “tissue product” will refer to any human or animal tissue that contains an extracellular matrix protein. “Tissue products” may include acellular or partially decellularized tissue matrices, as well as decellularized tissue matrices that have been repopulated with exogenous cells.

As used herein, the term “acellular tissue matrix” refers to an extracellular matrix derived from human or animal tissue, wherein the matrix retains a substantial amount of natural collagen, other proteins, proteoglycans, and/or glycoproteins needed to serve as a scaffold to support tissue regeneration. “Acellular tissue matrices” are different from purified collagen materials, such as acid-extracted purified collagen, which are substantially void of other matrix proteins and do not retain the natural micro-structural features of tissue matrix due to the purification processes. Although referred to as “acellular tissue matrices,” it will be appreciated that such tissue matrices may be combined with exogenous cells, including, for example, stem cells, or cells from a patient in whom the “acellular tissue matrices” may be implanted. A “decellularized adipose tissue matrix” will be understood to refer to an adipose-based tissue from which all cells have been removed to produce adipose extracellular matrix. “Decellularized adipose tissue matrix” can include intact matrix or matrix that has been further processed as discussed herein, including mechanical processing, formation of a sponge, and/or further processing to produce particulate matrix. As used herein, AAM refers to “acellular adipose matrix” from any source, and pAAM refers to “porcine-derived adipose matrix.”

“Acellular” or “decellularized” tissue matrices will be understood to refer to tissue matrices in which no cells are visible using light microscopy.

Various human and animal tissues may be used to produce products for treating patients. For example, various tissue products for regeneration, repair, augmentation, reinforcement, and/or treatment of human tissues that have been damaged or lost due to various diseases and/or structural damage (e.g., from trauma, surgery, atrophy, and/or long-term wear and degeneration) have been produced. Such products may include, for example, acellular tissue matrices, tissue allografts or xenografts, and/or reconstituted tissues (i.e., at least partially decellularized tissues that have been seeded with cells to produce viable materials).

A variety of tissue products have been produced for treating soft and hard tissues. For example, ALLODERM® and STRATTICE™ (LIFECELL CORPORATION, BRANCHBURG, N.J.) are two dermal acellular tissue matrices made from human and porcine dermis, respectively. Although such materials are very useful for treating certain types of conditions, materials having different biological and mechanical properties may be desirable for certain applications. For example, ALLODERM® and STRATTICE™ have been used to assist in the treatment of structural defects and/or to provide support to tissues (e.g., for abdominal walls or in breast reconstruction), and their strength and biological properties make them well suited for such uses. However, such materials may not be ideal for regeneration, repair, replacement, and/or augmentation of adipose-containing tissues, when the desired result is production of adipose tissue with viable adipocytes. Accordingly, the present disclosure provides tissue products that are useful for the treatment of tissue defects/imperfections involving adipose-containing tissues. The present disclosure also provides methods for producing such tissue products.

The tissue products may include adipose tissues that have been processed to remove at least some of the cellular components. In some cases, all, or substantially all cellular materials are removed, thereby leaving adipose extracellular matrix proteins. In addition, the products may be processed to remove some or all of the extracellular and/or intracellular lipids. In some cases, however, complete removal of extracellular and/or intracellular lipids can be damaging to the architecture and functions of the adipose matrix. For example, adipose tissues that are chemically or enzymatically treated for an extended period of time may have denatured or otherwise damaged collagen, or may be depleted of proteins needed for adipose regeneration. Accordingly, in some cases, the product contains a certain level of residual lipids. The remaining lipid content can be, for example, about 5%, 6%, 7%, 8%, 9%, or 10% by weight of the product. As described further below, the extracellular matrix proteins may be further treated to produce a three-dimensional porous, or sponge-like material, and the porous or sponge-like material may be further processed to produce an injectable product.

As noted, the tissue products of the present disclosure are formed at least partially from adipose tissues. The adipose tissues may be derived from human or animal sources. For example, human adipose tissue may be obtained from cadavers. In addition, human adipose tissue could be obtained from live donors (e.g., with autologous tissue). Adipose tissue may also be obtained from animals such as pigs, monkeys, or other sources. If non-primate animal sources are used, the tissues may be further treated to remove antigenic components such as 1,3-alpha-galactose moieties, which are present in pigs and other mammals, but not humans or primates. See Xu, Hui, et al., “A Porcine-Derived Acellular Dermal Scaffold that Supports Soft Tissue Regeneration: Removal of Terminal Galactose-α-(1,3)-Galactose and Retention of Matrix Structure,” Tissue Engineering, Vol. 15, 1-13 (2009), which is hereby incorporated by reference in its entirety. In addition, the adipose tissue may be obtained from animals that have been genetically modified to remove antigenic moieties.

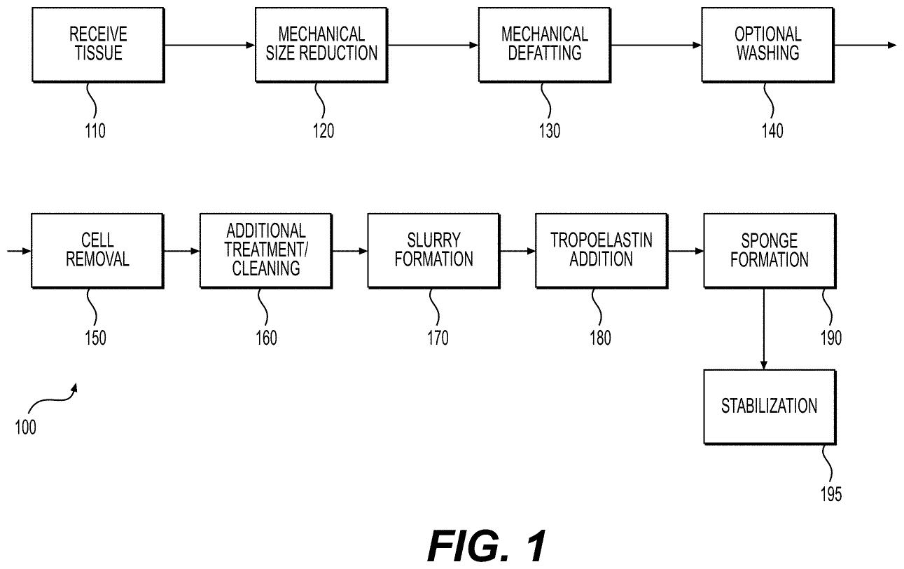

An exemplary process for producing the tissue products of the present disclosure is illustrated in FIG. 1. FIG. 1 provides a flow chart illustrating the basic steps that may be used to produce a suitable adipose tissue sponge. As shown, the process may include a number of steps, but it will be understood that additional or alternative steps may be added or substituted depending on the particular tissue being used, desired application, or other factors.

As shown, the process 100 may begin generally at Step 110, wherein tissue is received. The tissue may include a variety of adipose tissue types, including, for example, human or animal adipose tissue. Suitable tissue sources may include allograft, autograft, or xenograft tissues. When xenografts are used, the tissue may include adipose from animals including porcine, cow, dog, cat, domestic or wild sources, and/or any other suitable mammalian or non-mammalian adipose source.

The tissue may be harvested from animal sources using any desirable technique, but may be generally obtained using, if possible, aseptic or sterile techniques. The tissue may be stored in cold or frozen conditions or may be immediately processed to prevent any undesirable changes due to prolonged storage.

After receiving the tissue, the tissue may initially be subject to mechanical size reduction at Step 120 and/or mechanical defatting at Step 130. Mechanical size reduction may include gross or large cutting of tissue using manual blades or any other suitable grinding process.

Mechanical defatting at Step 130 may be important in the production of tissue. Specifically, to assist in lipid removal, the adipose may be subject to a variety of mechanical processing conditions. For example, the mechanical processing may include grinding, blending, chopping, grating, or otherwise processing the tissue. The mechanical processing may be performed under conditions that allow for a certain degree of heating, which may assist in liberating or removing lipids. For example, the mechanical processing may be performed under conditions that may allow the adipose tissue to heat up to 122° F. (50° C.), or between 42-45° C. for porcine adipose or somewhat lower temperatures for human adipose. The application of external heat may be insufficient to release the lipids; therefore, heat generated during mechanical disruption may be preferred to assist in lipid removal. In some examples, heating during mechanical processing may be a pulse in temperature rise and may be short in duration. This heat pulse may cause liquification of lipid released from broken fat cells by mechanical disruption, which may then cause efficient phase separation for bulk lipid removal. In an example, when processing a porcine adipose tissue, the temperature reached during this process is above 100° F. and may not exceed 122° F. (50° C.). The range of temperature reached can be adjusted according to the origin of the adipose tissue. For example, the temperature can be further lowered to about 80° F., 90° F., 100° F., 110° F., or 120° F. when processing tissues with less saturated fat, e.g., primate tissues. Alternatively, the process may be selected to ensure the adipose reaches a minimum temperature such as 80° F., 90° F., 100° F., 110° F., or 120° F.

In some cases, the mechanical defatting may be performed by mechanically processing the tissue with the addition of little or no washing fluids. For example, the tissue may be mechanically processed by grinding or blending without the use of solvents. Alternatively, when grinding the tissue requires moisture, for example to increase flowability or decrease viscosity, water may be used, including pure water or saline or other buffers including saline or phosphate buffered saline. In some examples, the tissue may be processed by adding a certain quantity of solvent that is biocompatible, such as saline (e.g., normal saline, phosphate buffered saline, or solutions including salts and/or detergents). Other solutions that facilitate cell lysis may also be appropriate, including salts and/or detergents.

After mechanical processing and lipid removal, the adipose may be washed at Step 140. For example, the tissue may be washed with one or more rinses with various biocompatible buffers. For example, suitable wash solutions may include saline, phosphate buffered saline, or other suitable biocompatible materials or physiological solutions. In an example, water may be used as a rinsing agent to further break the cells, after which phosphate buffered saline, or any other suitable saline solution, may be introduced to allow the matrix proteins to return to biocompatible buffers.

The washing may be performed along with centrifugation or other processes to separate lipids from the tissue. For example, in some embodiments, the material is diluted with water or another solvent. The diluted material is then centrifuged, and free lipids will flow to the top, while the extracellular matrix proteins are deposited as a pellet. The protein pellet may then be resuspended, and the washing and centrifugation may be repeated until a sufficient amount of the lipids are removed.

After any washing, the adipose may be treated to remove some or all cells from the adipose tissue as indicated at Step 150. The cell removal process may include a number of suitable processes. For example, suitable methods for removing cells from the adipose tissue may include treatment with detergents such as deoxycholic acid, polyethylene glycols, or other detergents at concentrations and times sufficient to disrupt cells and/or remove cellular components.

After cell removal, additional processing and/or washing steps may be incorporated, depending on the tissue used or ultimate structure desired, as indicated at Step 160. For example, additional washing or treatment may be performed to remove antigenic materials such as alpha-1,3-galactose moieties, which may be present on non-primate animal tissues. In addition, during, before, and/or after the washing steps, additional solutions or reagents may be used to process the material. For example, enzymes, detergents, and/or other agents may be used in one or more steps to further remove cellular materials or lipids, remove antigenic materials, and/or reduce the bacteria or other bioburden of the material. For example, one or more washing steps may be included using detergents, such as sodium dodecyl sulfate or Triton to assist in cell and lipid removal. In addition, enzymes such as lipases, DNAses, RNAses, alpha-galactosidase, or other enzymes may be used to ensure destruction of nuclear materials, antigens from xenogenic sources, residual cellular components and/or viruses. Further, acidic solutions and/or peroxides may be used to help further remove cellular materials and destroy bacteria and/or viruses, or other potentially infectious agents.

After removal of lipids and cellular components, the material may then be formed into a porous or sponge-like material. Generally, the extracellular matrix is first resuspended in an aqueous solvent to form a slurry-like material of AAM (acellular adipose matrix) as indicated at Step 170. A sufficient amount of solvent is used to allow the material to form a liquid mass that may be poured into a mold having the size and shape of the desired tissue product. The amount of water or solvent added may be varied based on the desired porosity of the final material. In some cases, the slurry-like material may have a solid concentration of about 2% to about 10% by weight, preferably about 2% to about 5%. The weight is to be understood to be the dry weight of AAM or TE (if added). So as an example, a 3% AAM slurry includes 3 g AAM in 100 mL liquid. After freeze drying the final material will weigh 3 g. Or if a slurry has 1% TE and 3% AAM it will include 1 g TE and 3 g AAM for each 100 mL slurry. In some cases, the resuspended extracellular matrix may be mechanically treated by grinding, cutting, blending or other processes one or more additional times, and the treated material may be centrifuged and resuspended one or more times to further remove cellular material or lipids (if needed) and/or to control the viscosity of the extracellular matrix.

In some cases, tropoelastin may be added to the material before formation of the sponge, as indicated at Step 180. A variety of suitable tropoelastins may be selected. For example, the tropoelastin can be provided in a form suitable for mixing into the AAM slurry. For example, the tropoelastin may be formed from a tropoelastin solution into an elastic material, a viscoelastic material, or hydrogel, by treating with chemical processes and/or heat. The tropoelastin, after treatment to form an elastic, viscoelastic, or hydrogel, can then be cut or otherwise processed to allow mixing with previously prepared acellular adipose matrix.

Exemplary processes for making tropoelastins are discussed further below under a separate heading.

The tropoelastin can be added to the AAM slurry at a range of suitable concentrations. For example, the amount of tropoelastin can be selected to impart desired mechanical and/or biologic properties. For example, the tropoelastin can be mixed with the AAM at a weight percentage of 0.1 to 50%, or from 0.5-3%, 1-5%, 0.5-2%, 10%-75%, or other suitable values.

The tropoelastin can be produced and mixed with the AAM sponge slurry of Step 170, in a number of forms, including, for example, the tropoelastin being mixed into the AAM slurry while the tropoelastin is the form of a dry powder, solution, slurry or a hydrogel. For example, a TE hydrogel can be incorporated into an AAM slurry to a desired weight. An exemplary TE hydrogel can be made by cross-linking a human TE using EDC, BS3, or other cross-linker. Alternatively, the TE can be dried by freeze-drying and milled to produce a dry powder, and the powder can be incorporated into an AAM slurry.

The amount of TE can vary based on a number of factors, including desired mechanical or biological properties. For example, the TE can be incorporated into a slurry to form 0.1-10% weight of the slurry with AAM at 0.1-10% of the slurry. In some cases, the TE and AAM are included at a desired ratio such as TE:AAM in slurry of 1:3, 1:4, 1:5 (by weight), or other suitable ranges.

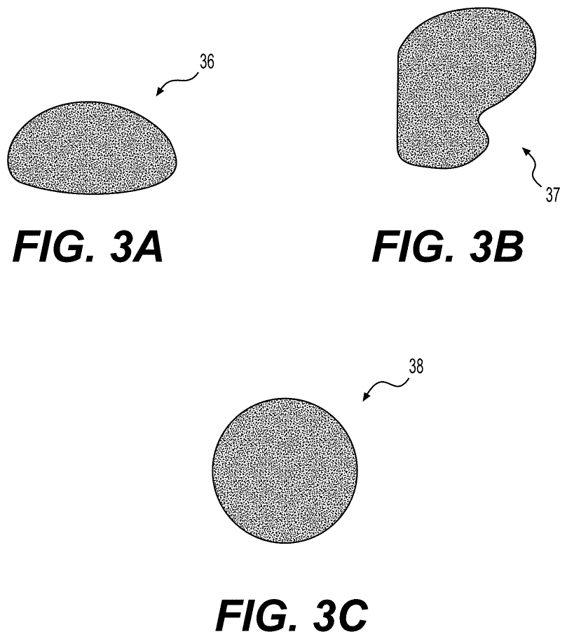

After mixing tropoelastin with the AAM and suspending the material, the material is placed in a container or mold to form the porous, sponge-like product, as indicated at Step 190. Generally, the porous or sponge-like material is formed by drying the material to leave a three-dimensional matrix with a porous structure. In some embodiments, the material is freeze-dried. Freeze-drying may allow production of a three-dimensional structure that generally conforms to the shape of the mold, as shown in FIG. 3. The specific freeze drying protocol may be varied based on the solvent used, sample size, and/or to optimize processing time. One suitable freeze-drying process may include cooling the material; holding the samples at the cooled temperature and further cooling down the sample to ensure complete freezing; applying a vacuum; and raising the temperature in one or several steps. The freeze-dried samples may then be removed from the freeze-dryer and packaged in foil pouches under nitrogen.

After formation of a solid or sponge, the material may optionally be stabilized, as indicated at Step 195. In some cases, the stabilization may include additional processes such as cross-linking, treatment with dehydrothermal (DHT) processes, or other suitable stabilization methods. For example, generally, a mechanically processed tissue, when formed into a porous matrix, may form a more putty- or paste-like material when it is implanted in a body, becomes wet, or is placed in a solution. Therefore, the desired shape and size may be lost. In addition, the porous structure, which may be important for supporting cell attachment, tissue growth, vascular formation, and tissue regeneration, may be lost. Accordingly, the material may be further processed to stabilize the size, shape, and structure of the material.

In some embodiments, the TE-AAM material is cross-linked for stabilization. Exemplary cross-linking processes may include contacting a freeze-dried material, produced as discussed above, with glutaraldehyde or EDC.

In some embodiments, the material is cross-linked after freeze drying. However, the material could also be cross-linked before or during the freeze-drying process. Cross-linking may be performed in a variety of ways. In one embodiment, cross-linking is accomplished by contacting the material with a cross-linking agent such as glutaraldehyde, genepin, carbodiimides (e.g., 1-ethyl-3-(3-dimethylaminopropyl) carbodiimide hydrochloride (EDC)), and diisocyantes.

In addition, cross-linking may be performed by heating the material in a vacuum (DHT). For example, in some embodiments, the material may be heated to between 70° C. to 120° C., or between 80° C. and 110° C., or to about 100° C., or any values between the specified ranges in a reduced pressure or vacuum. DHT treatment may also be followed by chemical cross-linking to produce a desired mechanical property (e.g., DHT followed by EDC or other chemical cross-linking). In addition, other cross-linking processes, or combination of processes, may be used to produce any of the disclosed products, including ultraviolet irradiation, gamma irradiation, and/or electron beam irradiation. In addition, a vacuum is not needed but may reduce cross-linking time. Further, lower or higher temperatures could be used as long as melting of the matrix proteins does not occur and/or sufficient time is provided for cross-linking.

In various embodiments, the cross-linking process may be controlled to produce a tissue product with desired mechanical, biological, and/or structural features. For example, cross-linking may influence the overall strength of the material, and the process may be controlled to produce a desired strength. In addition, the amount of cross-linking may affect the ability of the product to maintain a desired shape and structure (e.g., porosity) when implanted. Accordingly, the amount of cross-linking may be selected to produce a stable three-dimensional shape when implanted in a body, when contacted with an aqueous environment, and/or when compressed (e.g., by surrounding tissues or materials).

Excessive cross-linking may change the extracellular matrix materials. For example, excessive cross-linking may damage collagen or other extracellular matrix proteins. The damaged proteins may not support tissue regeneration when the tissue products are placed in an adipose tissue site or other anatomic location. In addition, excessive cross-linking may cause the material to be brittle or weak. Accordingly, the amount of cross-linking may be controlled to produce a desired level of stability, while maintaining desired biological, mechanical, and/or structural features.

In some cases, rather than providing the disclosed tissue products as a sponge, the products can be formed into particulate or flowable and injectable materials. Such compositions can be used for applications such as bulking (e.g., to smooth wrinkles, increase tissue size in lips or other structures, or to improve shape or features of other anatomic structures.

Injectable compositions can be formed in a number of suitable ways. In particular a tropoelastin component can be mixed with an adipose tissue matrix product to produce an injectable composition in a number of ways. For example, in some cases, the adipose matrix sponge, with tropoelastin, is produced as illustrated in FIG. 1 up to Step 195. After stabilization, the sponge can be particulated, by mechanical means such as cutting or grinding, and the particulate, if desired can be sorted or size-selected to produce a desired size distribution range.

Alternatively, in some cases, AAM sponge can be formed and particulated. Subsequently, the particulate AAM can be mixed with particulate tropoelastin, and the composition can be used as an injectable. Tropoelastin can be added at a range of suitable percentage (e.g., 10-75%).

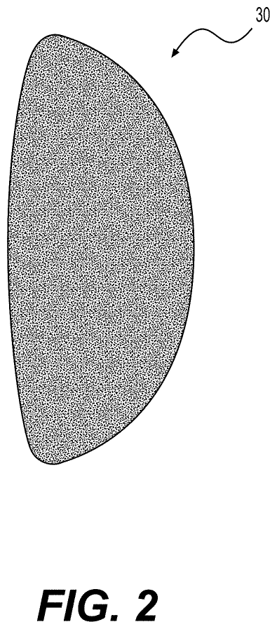

The devices produced using the above-discussed methods can have a variety of configurations. For example, FIG. 2 is a side view of a biologic breast implant 30 formed of a tropoelastin and adipose tissue matrix. The implant can include a variety of suitable breast implant shapes, contours, or projections. Further, it should be appreciated that a variety of shapes can be used, including rounded, irregular, concentric spheroid, or concentric irregular 3-D shapes, or custom-formed implants. For example, FIGS. 3A-3C illustrate exemplary shapes for implants produced using the disclosed methods, including tear-drop implants 36 (FIG. 3A), irregular implants 37 (FIG. 3B), and/or spherical implants 38 (FIG. 3C).

The device 30, 36-38 can have a variety of sizes. But as noted above, the methods provided herein can provide advantages by allowing production of adipose implants having large sizes that can match those of conventional breast implants or tissue expanders. For example, using the methods discussed herein, implants having at least one dimension of 5 cm or greater can be produced. In other cases, the devices have a dimension of at least 6 cm, at least 7 cm, at least 8 cm, at least 10 cm, or larger.

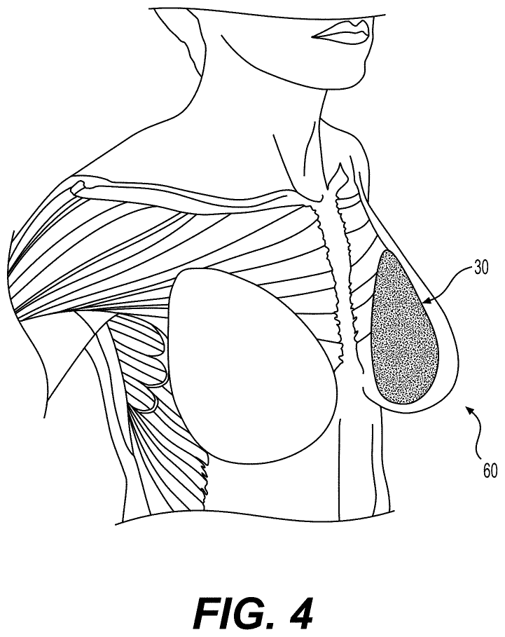

Also disclosed herein are methods for treating a breast by implanting the tissue product. Accordingly, FIG. 4 illustrates implantation of a system for surgical breast procedures. The method can first include identifying an anatomic site within a breast 60. (As used herein, “within a breast” will be understood to be within mammary tissue, or within or near tissue surrounding the breast such as tissue just below, lateral or medial to the breast, or beneath surrounding tissues including, for example, under chest (pectoralis) muscles, and will also include implantation in a site in which part or all of the breast has already been removed via surgical procedure). The site can include, for example, any suitable site needing reconstruction, repair, augmentation, or treatment. Such sites may include sites in which surgical oncology procedures (mastectomy, lumpectomy) have been performed, sites where aesthetic procedures are performed (augmentation or revisions augmentation), or sites needing treatment due to disease or trauma.

Further provided herein are methods of treatment comprising the steps of selecting a tissue site and implanting the tissue products disclosed herein into the tissue site. The methods can include implanting the treatment device in or proximate to a wound or surgical site and securing at least a portion of the treatment device to tissue in or near the treatment site. The tissue product may be implanted behind the tissue site to bolster, reposition, or project the native tissue outward.

Also provided herein are methods of treatment comprising selecting a tissue site within a breast; implanting a device within the tissue site; and allowing tissue to grow within the acellular adipose tissue matrix. In one embodiment, the device comprises a synthetic breast implant or tissue expander and an acellular adipose tissue matrix surrounding the breast implant or tissue expander. The method can further include removing the breast implant or tissue expander and implanting an additional acellular adipose tissue matrix within a void formed by removal of the breast implant or tissue expander.

The tissue products described herein can be used to treat a variety of different anatomic sites. For example, as discussed throughout, the tissue products of the present disclosure are produced from adipose tissue matrices and can be used for treatment of breasts. In some cases, the tissue products can be implanted in other sites, including, for example, tissue sites that are predominantly or significantly adipose tissue. In some cases, the tissue sites can include a breast (e.g., for augmentation, replacement of resected tissue, or placement around an implant). In addition, any other adipose-tissue containing site can be selected. For example, the tissue products may be used for reconstructive or cosmetic use in the breast, face, buttocks, abdomen, hips, thighs, or any other site where additional adipose tissue having structure and feel that approximates native adipose may be desired. In any of those sites, the tissue may be used to reduce or eliminate wrinkles, sagging, or undesired shapes.

The process of the invention and the elastic material formed from this are particularly useful in tissue bulking applications, for example, applications where there is a need to cosmetically enhance or improve appearance (for example, plumping of lips, filling-in of nasolabial folds, reduction of wrinkles or other tissue enhancements), or medical applications where there is a need to support a congenital defect, or defect caused by disease or surgical resection.

In some cases, the product comprises an injectable composition which includes particulate AAM and TE contained in a carrier solution. If in a particular form, the material can be configured to allow a desired flowability or injectability. For example, various carriers can be added at a certain percentage to allow flowability. Suitable carriers could include solutions (e.g., PBS), hydrogels, hyaluronic acid or derivatives of hyaluronic acid, gelatin, or other biocompatible flowable materials.

### Exemplary Methods of Producing Tropoelastin:

As noted above, the tropoelastin may be provided in a number of forms, such as an elastic, viscoelastic, hydrogel, or other materials. Generally an “elastic material” is not a free-flowing liquid. It may be a gel, paste, solid, or other phase that significantly lacks the properties of flow. An “elastic material” generally returns to a particular shape or conformation after a force such as compression or extension that has been applied to it has been withdrawn. “Elastic material” is also referred to as a resiliently compressible and extendible, mechanically durable, or pliable material of relatively low hysteresis. This material may be referred to as stretchable, tensile, resilient or capable of recoil.

It will be understood that “tropoelastin” generally means a peptide that includes or consists of a sequence that is the same as or similar to a hydrophilic domain of tropoelastin. A hydrophilic domain has a sequence that is typically rich in lysine and alanine residues. These domains often consist of stretches of lysine separated by 2 or 3 alanine residues such as AAAKAAKM (SEQ ID NO: 1). Other hydrophilic domains do not contain the poly-alanine tract but have lysine near a proline instead. In contrast, tropoelastin hydrophobic domains are rich in non-polar amino acids especially glycine, valine, proline and alanine and often occur in repeats of 3 to 6 peptides such as GVGVP (SEQ ID NO: 2), GGVP (SEQ ID NO: 3) and GVGVAP (SEQ ID NO: 4).

Examples of tropoelastin that could be used with the present AAMs are those that consist of a hydrophilic domain or a homolog thereof, and those that include a hydrophilic domain or homolog and part or all of a hydrophobic domain. Some examples are set out below:

(SEQ ID NO: 5)
GGVPGAIPGGVPGGVFYP,
(SEQ ID NO: 6)
GVGLPGVYP,
(SEQ ID NO: 7)
GVPLGYP,
(SEQ ID NO: 8)
PYTTGKLPYGYGP,
(SEQ ID NO: 9)
GGVAGAAGKAGYP,
(SEQ ID NO: 10)
TYGVGAGGFP;
(SEQ ID NO: 11)
KPLKP,
(SEQ ID NO: 12)
ADAAAAYKAAKA,
(SEQ ID NO: 13)
GAGVKPGKV,
(SEQ ID NO: 14)
GAGVKPGKV,
(SEQ ID NO: 15)
TGAGVKPKA,
(SEQ ID NO: 16)
QIKAPKL,
(SEQ ID NO: 17)
AAAAAAAKAAAK,
(SEQ ID NO: 18)
AAAAAAAAAAKAAKYGAAAGLV,
(SEQ ID NO: 19)
EAAAKAAAKAAKYGAR,
(SEQ ID NO: 20)
EAQAAAAAKAAKYGVGT,
(SEQ ID NO: 21)
AAAAAKAAAKAAQFGLV,
(SEQ ID NO: 22)
GGVAAAAKSAAKVAAKAQLRAAAGLGAGI,
(SEQ ID NO: 23)
GALAAAKAAKYGAAV,
(SEQ ID NO: 24)
AAAAAAAKAAAKAA,
(SEQ ID NO: 25)
AAAAKAAKYGAA,
(SEQ ID NO: 26)
CLGKACGRKRK.

“Tropoelastin” may have a sequence that is the same as the entry shown in GenBank entry AAC98394. Other tropoelastin sequences including a hydrophilic domain are known in the art, including, but not limited to, CAA33627 (Homo sapiens), P15502 (Homo sapiens), AM42271 (Rattus norvegicus), AAA42272 (Rattus norvegicus), AAA42268 (Rattus norvegicus), AAA42269 (Rattus norvegicus), AAA80155 (Mus musculus), AAA49082 (Gallus gallus), P04985 (Bos taurus), ABF82224 (Danio rerio), ABF82222 (Xenopus tropicalis), P11547 (Ovis aries).

“Tropoelastin” may also be a fragment of these sequences provided that the fragment includes at least part of a hydrophilic domain as discussed above. An example is amino acids 27 to 724 of AAC98394. The tropoelastin used in the present disclosure may include human tropoelastin or a selected domain of human tropoelastin.

Tropoelastin may also include a homolog of a peptide having a sequence such as described above, in particular AAC98394, or be a homolog of a peptide having a sequence such as described above, or be a fragment of a homolog of a peptide having a sequence such as described above. Herein “homolog” refers to a protein having a sequence that is not the same as, but that is similar to, a reference sequence. It also has the same function as the reference sequence, for example, a capacity to form an elastic material when a solution of the homolog is manipulated to adjust alkalinity, temperature or salt concentration as discussed herein.

In certain embodiments the homolog has at least 60% homology to a peptide such as described above, in particular AAC98394 or a fragment of a peptide such as described above that includes at least part of a hydrophilic domain.

It will be understood that “tropoelastin” may be natural or recombinant.

There exists a subset of temperature, alkalinity, and salt concentration conditions within which a solution of tropoelastin can be made to form an elastic material. In one embodiment there is provided a process for producing an elastic material from tropoelastin including heating a solution of tropoelastin having an alkaline pH to form an elastic material from the tropoelastin in the solution.

In other embodiments there is provided a process for producing an elastic material from tropoelastin including providing an alkaline pH to a solution of tropoelastin having a temperature of about 37° C. to form an elastic material from the tropoelastin in the solution.

In other embodiments there is provided a process for producing an elastic material from tropoelastin including providing an alkaline pH to a solution of tropoelastin and allowing the temperature of the solution to increase to about 37° C. to form an elastic material from the tropoelastin in the solution.

In other embodiments there is provided a process for producing an elastic material from tropoelastin including adding tropoelastin to a solution having an alkaline pH and a temperature of about 37° C. to form an elastic material from the tropoelastin in the solution.

In other embodiments there is provided a process for producing an elastic material from tropoelastin including adding tropoelastin to a solution having an alkaline pH and allowing the temperature of the solution to increase to about 37° C. to form an elastic material from the tropoelastin in the solution.

In other embodiments there is provided a process for producing an elastic material from tropoelastin including adjusting the salt concentration of a solution of tropoelastin having an alkaline pH and a temperature of about 37° C. to form an elastic material from the tropoelastin in the solution.

Generally a solution with a tropoelastin concentration greater than about 1.5 mg/mL is capable of forming an elastic material of desirable integrity although lesser concentrations are also useful. In most applications the solution concentration is less than about 300 mg/mL. Therefore, a solution of tropoelastin having a concentration from about 1.5 mg/mL to about 300 mg/mL is preferable. More preferably, a solution of tropoelastin having a concentration between about 10 mg/mL to about 300 mg/mL is used. Most preferably, a solution of tropoelastin having a concentration of between about 10 mg/mL to about 200 mg/mL is used.

It has been determined that a pH of about pH 7.5 or more is sufficient to cause an elastic material to form from the tropoelastin in the solution. The pH is generally kept from exceeding about pH 13 as above this the elastic material is less well formed. More preferably a pH of between about pH 9 and pH 13 is desirable. However, most preferably a pH of between about pH 10 and pH 11 is used. Other pH measures that could be used include 8.0, 8.5, 9.5, 10, 10.5, and 11.5.

Alkalinity can be adjusted by a number of approaches including 1) directly adding a pH increasing substance to a solution of tropoelastin, 2) by mixing a solution containing sufficient amounts of a pH increasing substance to cause it to be alkaline with a solution of tropoelastin. The pH increasing substance could be a base, buffer, proton adsorbent material. Examples including Tris base, NH₄OH and NaOH have been found to be useful as pH increasing or controlling substances.

Where the pH is alkaline and less than about 9.5, salt may be required to form the elastic tropoelastin material useful in forming sponges of the invention. Where salt is used, the concentration is generally more than 25 mM and may be up to 200 mM. Preferably, the salt concentration is between about 100 mM and 150 mM. More preferably, the salt concentration is about 150 mM. In particular, the inventors have found that as pH decreases (and yet remains alkaline) below pH 10, salt is required to cause formation of the elastic material and the amount of salt required increases as pH decreases. So for example, at about pH 9 to 10, salt is required, for example a salt concentration equivalent to about 60 mM should be provided to the solution. In some embodiments, the solution is to have an osmolarity equivalent to that of mammalian isotonic saline (150 mM) or less. In other embodiments, the solution is to have an osmolarity greater than 150 mM. The salt concentration may also be 0 mM.

The salt concentration of the solution may be controlled by adding salt, including any ionic compound, monovalent or divalent ions, or low molecular weight species capable of affecting the osmolality of the solution. For instance, NaCl, KCl, MgSO4, Na2CO3 or glucose may be used. A preferred salt is NaCl.

A method of forming an elastic material from solution of tropoelastin can include:

(1) providing a solution of tropoelastin;

(2) adjusting the pH of the solution to form a solution having alkaline pH, to cause the tropoelastin in the solution to precipitate;

(3) removing the precipitate;

(4) adding the removed precipitate to a solution having a substantially non alkaline pH, and/or a substantially lowered temperature, to cause the precipitate to disperse into the solution; and

(5) allowing the temperature of the solution to increase to about 37° C. to form an elastic material from the tropoelastin in the solution.

In one embodiment the temperature of the solution is preferably between about 4° C. to about 37° C. at step (2) and less than about 4° C. at step (4). Further, in one embodiment the pH of the solution is preferably at least about pH 9 at step (2) and less than about pH 9 at step (4). The pH may be as low as about pH 7.5 at step (4). Further still, in one embodiment the salt concentration of the solution is preferably between about 0 mM and 200 mM.

In other embodiments a process for producing an elastic material from tropoelastin includes heating a solution of tropoelastin having an alkaline pH that is less than 10 and a salt concentration of 150 mM or less to form an elastic material from the tropoelastin in the solution. These embodiments are particularly preferable for in vivo applications since the pH of the solution of tropoelastin and the elastic material is closer to mammalian pH.

In further embodiments a process for producing an elastic material from tropoelastin includes adjusting the salt concentration of a solution of tropoelastin having an alkaline pH and allowing the temperature of the solution to increase to about 37° C. to form an elastic material from the tropoelastin in the solution.

It has been determined that a temperature of around 37° C. is preferable to cause an elastic material to form from the tropoelastin in the solution. However, in certain embodiments a temperature of less than 37° C. may be used. Generally the temperature is greater than 4° C. It is generally less than 42° C.

The solution may be heated by providing the solution in or on a mammalian tissue and allowing the heat transfer from the tissue to increase the temperature of the solution, or by irradiating the tissue.

Alternatively, the solution may be heated by contacting the solution with an inanimate surface and heating the surface. The inanimate surface may be provided on a mold or cast for providing the elastic material formed by the method with a pre-defined shape or conformation

Where heating of the solution is provided to trigger the formation of the elastic material (e.g. where appropriate pH and/or salt conditions have been provided), the solution is generally stored at temperatures below 30° C., preferably about 4° C., until it is required for forming an elastic material to be used in a method for forming a sponge of the invention.

In certain embodiments the elastic material formed from a solution of tropoelastin by a process described above may be cross-linked with an agent capable of cross-linking the side chains of residues (e.g., lysine residues) of tropoelastin. In certain applications described below, it is useful to cross-link side chains when the elastic material has been formed as this provides further properties to the elastic material. Specifically, in comparison to the elastic material formed in the absence of cross-linker, the use of a cross-linker such as glutaraldehyde gives an elastic material that is stiffer, denser, tougher, and therefore likely more biostable in vivo. Cross-linked material may be preferable over the non-cross-linked elastic material for more demanding tissue restoration applications or when compliance with the surrounding natural tissue is non-essential.

It is contemplated that any cross-linking agent that can be used to form elastin, whether naturally or artificially, may be used. Examples include lysyl oxidase, transglutaminase, carbodiimides (e.g., 1-ethyl-3-(3-dimethylaminopropyl) carbodiimide hydrochloride (EDC), glutaraldehyde, genipin and amine-reactive cross-linkers such as bis(sulfosuccinimidyl)suberate (BS3). In one embodiment, the cross-linking agent is glutaraldehyde and is used at a concentration of about 0.001 w/v % solution to about 0.5 w/v % solution or BS3 at 1 mM to 100 mM.

In some cases, the tropoelastin (sometimes referred to herein as TE) can be cross-linked by heating for example, heating to 120-180° C. for 10-24 hours can allow suitable crosslinking.

### Example: Effect of Cross-Linking on Adipogenesis

3D acellular adipose matrix (AAM) sponges reduce seroma, hematoma, and scar formation, as well as promote adipogenesis. The mechanical properties of the sponges must be able to properly withstand the compressive forces in the body. In order to improve the mechanical strength and resilience of 3D AAM sponges, the sponges were altered by chemical crosslinking (e.g., 1-Ethyl-3-(3-dimethylaminopropyl)carbodiimide; EDC). Yet there is often a tradeoff between biological response and mechanical strength achieved by crosslinking. Therefore, a subcutaneous nude rat model was used to assess the biological response to the crosslinked sponges.

Porcine acellular adipose matrix (pAAM) slurry was prepared, freeze dried, and by DHT crosslinked at 80° C. for 24 hours. Some samples were also crosslinked in either 0.016% or 0.125% EDC. N-hydroxysuccinimide (NHS) was also added at a 5:3 EDC:NHS ratio. Sponges were then terminally sterilized by e-beam. Sponges with a thickness of approximately 5 mm were cut with a 10 mm biopsy punch and then implanted subcutaneously into nude rats (n=5). At 4 weeks, the explants were cut in half, with one half fixed in 10% formalin for Masson's trichrome staining and the other half fixed in sucrose.

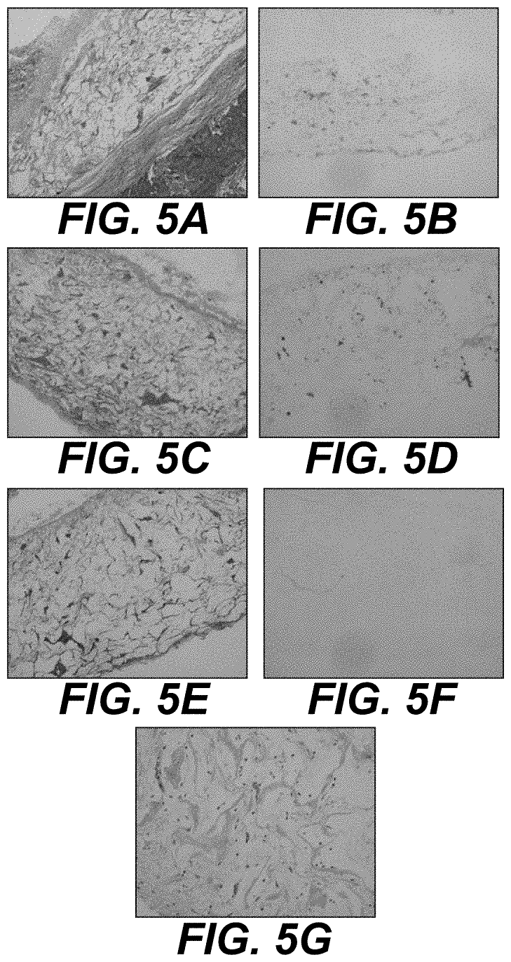

By 4 weeks the uncrosslinked sponges exhibited cell ingrowth, vascularization, and adipogenesis (FIGS. 5 A and B). In contrast, the 0.125% EDC crosslinked sponges did not exhibit any adipocytes by Oil Red 0 staining (FIGS. 5 E and F). Sponges with an intermediate amount of crosslinking (0.016%) showed a level of adipocytes that was intermediate to the levels found in the 0.125% and uncrosslinked sponges (FIGS. 5 C and D). However, trichrome staining revealed extensive cell ingrowth and vascularization for all sponge types (FIGS. 5 A, C, E, and G). This suggests that adipogenesis may be merely delayed by EDC crosslinking, not prevented entirely.

Overall, as EDC crosslinking was increased there was a concomitant decrease in adipogenesis, as evidenced by trichrome and Oil Red 0 staining. All three sponge types promoted cell ingrowth and vascularization regardless of the crosslinking conditions.

### Example: Effect of Processing on Mechanical Properties

AAM sponges should generally have mechanical properties to properly withstand the compressive forces in the body. In order to improve the mechanical strength and resilience of 3D AAM sponges, the sponges were altered by (1) changing the AAM solid content, or (2) chemical crosslinking (e.g., 1-Ethyl-3-(3-dimethylaminopropyl)carbodiimide; EDC).

AAM slurry was prepared with either a 3% or 4% solid content in 20% PBS. The slurry was then freeze dried to form sponges, followed by DHT crosslinking at 80° C. for 24 hours. The sponges were formed of slurry with 3 or 4% solid content, and if crosslinked with EDC, were incubated at room temperature for 4 hours in either 0.03% or 0.1% EDC in MES buffer. N-hydroxysuccinimide (NHS) was also added to the buffer at a 5:3 EDC:NHS ratio. Following crosslinking, the sponge was washed twice with PBS. The sample solid content and EDC amount were as follows:

| Sample # | Name |
|---|---|
| 1 | 3% AAM |
| 2 | 4% AAM |
| 3 | 3% AAM 0.03% EDC |
| 4 | 4% AAM 0.03% EDC |
| 5 | 3% AAM 0.1% EDC |
| 6 | 4% AAM 0.1% EDC |

Compression testing was performed on sponges hydrated with PBS to assess compressive strength at 50% strain, percent shape recovery following compression, and modulus. Here, modulus is defined as the slope of the linear region of the force-displacement curve. Tensile testing was performed to assess elasticity with sponge strips that were hydrated with PBS and then gently squeezed to remove excess liquid.

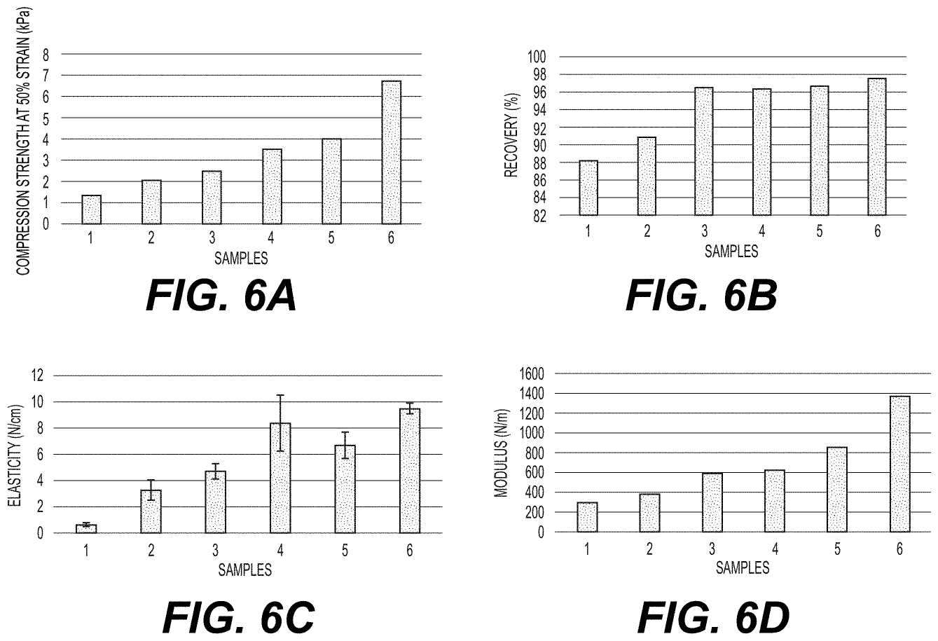

There was an overall linear trend for compressive strength, elasticity, and modulus as EDC percentage was increased (FIG. 6A, C, D). For each EDC crosslinking condition, the 4% AAM sponge was stronger than its 3% counterpart.

The 4% AAM sponge with 0.1% EDC (Sample 6) exhibited the highest strength by these parameters. FIG. 6B shows that both the 0.03% and 0.1% EDC crosslinking conditions on average similarly improved shape recovery 7.2% over the uncrosslinked versions.

Increasing the solid content from 3% to 4% increased mechanical strength of the sponges.

EDC crosslinking the sponges further increased mechanical strength, with the higher EDC concentration (0.1%) resulting in stronger sponges than the lower EDC concentration (0.03%).

### Example: Effect of Processing on Adipogenesis and Mechanical Properties

In another sponge composition, 10 mg/ml tropoelastin in PBS was crosslinked with 10 mM bis(sulfosuccinimidyl)suberate (BS3) at 37° C. for 18 hours to form a hydrogel. The tropoelastin hydrogel was then washed in PBS, cut, and incorporated into the AAM slurry to a final concentration of 1% w/v (1% of the slurry with a 3% AAM content). Alternatively, the tropoelastin hydrogel was freeze dried, cut, cryomilled to form a powder and incorporated into the AAM slurry to a final concentration of 1%. The tropoelastin:AAM sponges were then freeze dried and crosslinked as described above (DHT or EDC).

Compression testing was performed on sponges hydrated with PBS to assess compressive strength at 50% strain, percent shape recovery following compression, and modulus. The biological response of the tropoelastin/AAM sponges were also tested in vivo. Sponges were terminally sterilized by E-beam at 15-25 kGy. Sponges with a thickness of approximately 5 mm were cut with a 10 mm biopsy punch, washed in saline, and then implanted subcutaneously into nude rats (n=5). At 8 and 16 weeks, the explants were fixed in 10% formalin for Masson's trichrome staining.

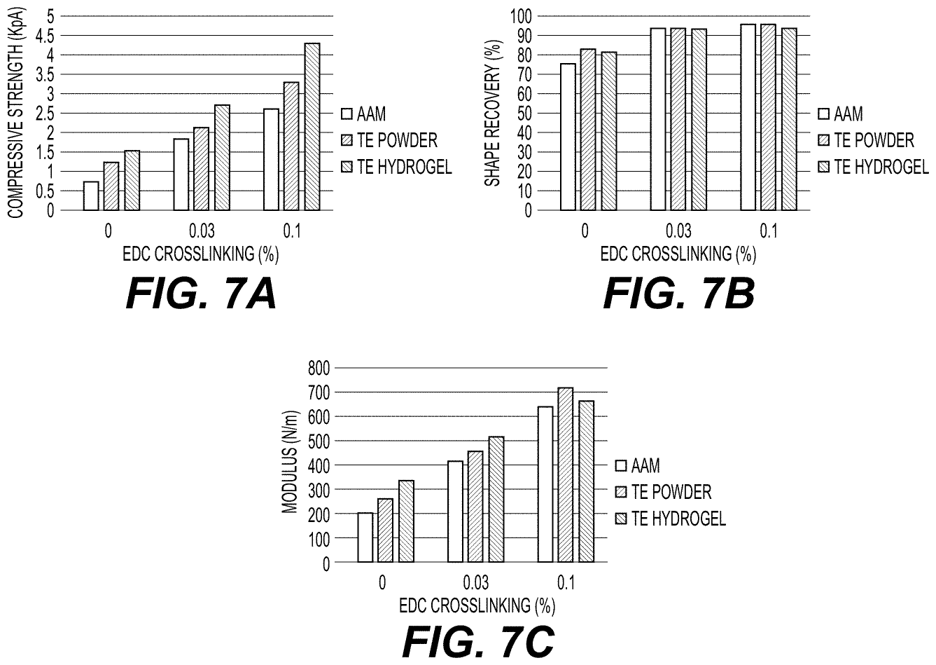

There was an overall linear trend for compressive strength and modulus as EDC percentage was increased (FIG. 7A, C). The addition of the tropoelastin hydrogel resulted in an approximate 2-fold increase in compressive strength as compared to the AAM control, and the tropoelastin powder was intermediate. The 3% AAM sponge with 0.1% EDC and the tropoelastin hydrogel exhibited the highest strength by these parameters. FIG. 7B shows that both the 0.03% and 0.1% EDC crosslinking conditions on average similarly improved shape recovery approximately 10% over the uncrosslinked versions.

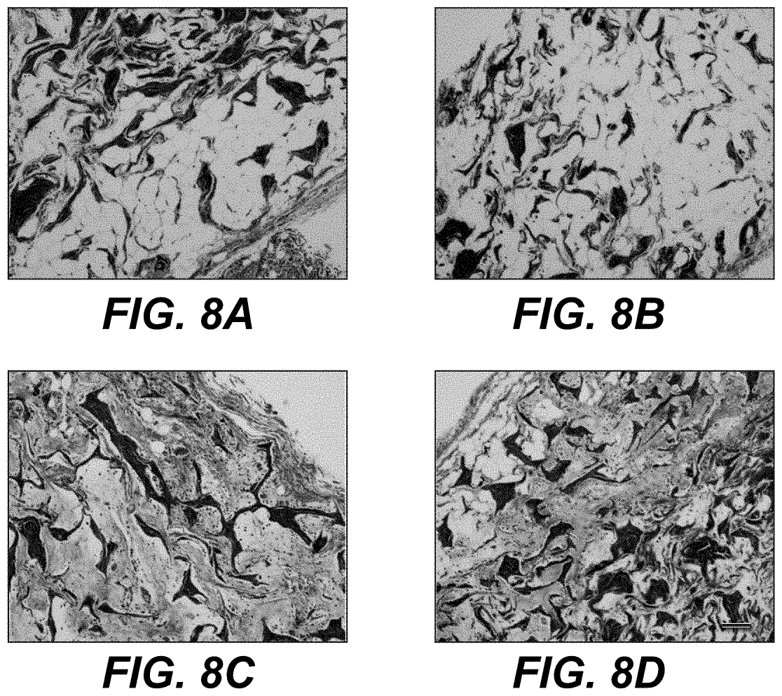

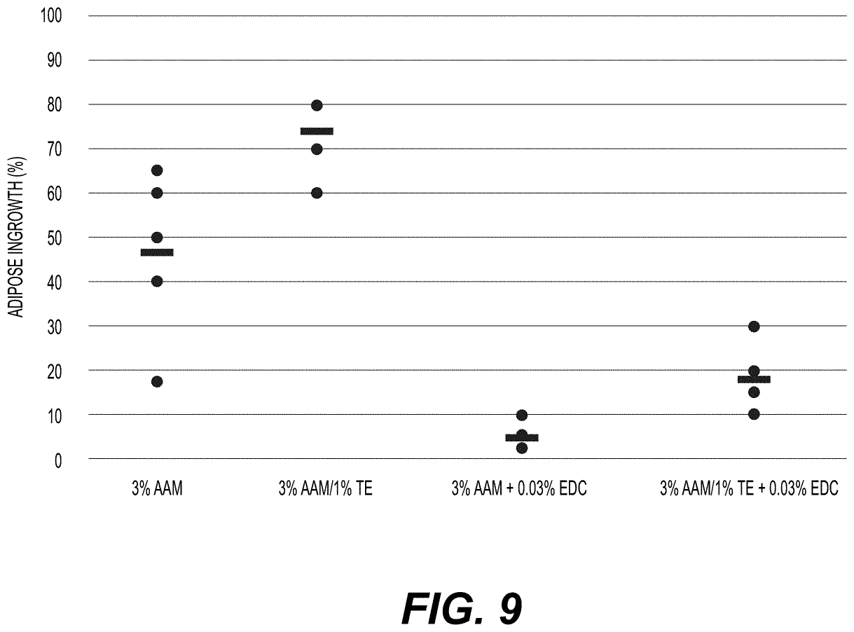

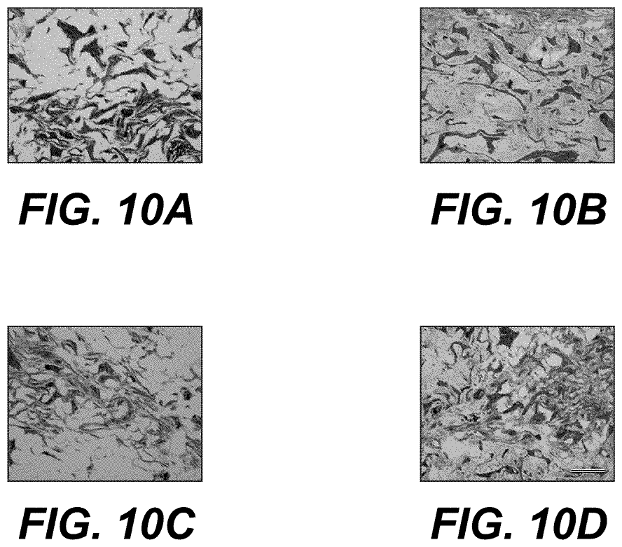

Evaluation of Masson's Trichrome stained explants revealed evidence of extensive cell repopulation and vascularization at both time points for 8 and 16 weeks in nude rat (FIGS. 8A-8D (8 weeks) and FIGS. 10A-10D (16 weeks)). The inflammatory cell infiltration was higher in the crosslinked groups as compared to the uncrosslinked control at both time points. EDC crosslinking at 0.03% significantly decreased the average adipose tissue percentage at both time points as compared to the uncrosslinked control (ANOVA; Tukey post hoc; p<0.05). FIG. 9 demonstrates that with the addition of the tropoelastin hydrogel the average percentage of adipose tissue at 8 weeks was significantly increased as compared to all groups (74±8.9; ANOVA; Tukey post hoc; p<0.05). Likewise, adding tropoelastin to the 0.03% EDC crosslinked AAM sponges significantly increased the average percentage of adipose tissue as compared to the crosslinked groups, but was still lower than its uncrosslinked counterpart (ANOVA; Tukey post hoc; p<0.05).

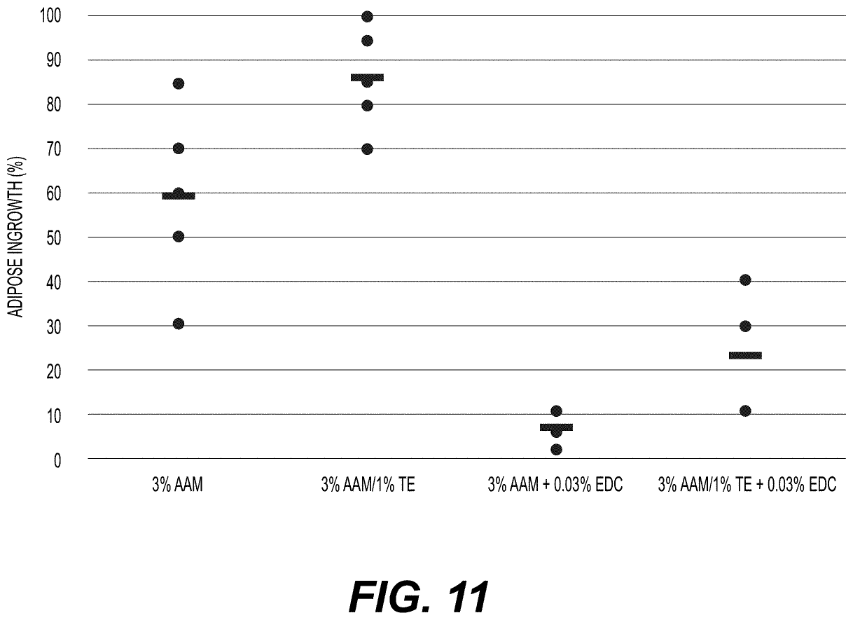

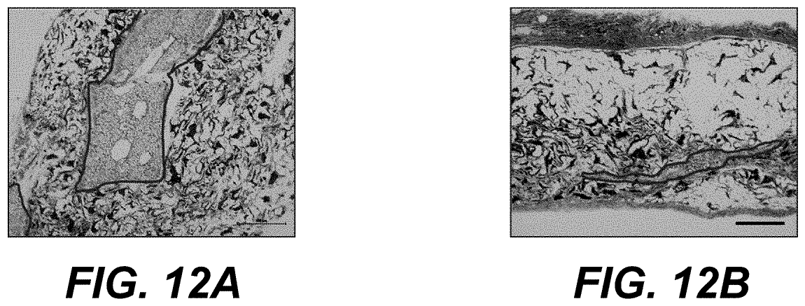

By 16 weeks the overall percentage of adipose tissue for all groups increased. However, the adipocyte coverage for the crosslinked groups of 3% pAAM was still below 10%, indicating that the adipogenic potential of pAAM is compromised by crosslinking at EDC concentrations of at least 0.03%. Tropoelastin addition significantly improved the adipose percentage at 16-weeks when comparing to its corresponding uncrosslinked 3% pAAM samples (ANOVA; Tukey post hoc; p<0.05) (FIG. 11). The tropoelastin hydrogel persisted in the sponge, and some remained by 16 weeks (FIGS. 12A-B). The crosslinked pAAM with tropoelastin was not significantly different from the other crosslinked groups at 16 weeks (ANOVA; Tukey post hoc; p<0.05).

The tropoelastin hydrogel improved both the mechanical and biological properties of the AAM sponge. The tropoelastin powder was intermediate in improving the compressive strength and modulus. EDC crosslinking the sponges improved mechanical strength, with the higher EDC concentration (0.1%) resulting in stronger sponges than the lower EDC concentration (0.03%). EDC crosslinking at 0.03% decreased adipose tissue ingrowth significantly. Incorporating the TE hydrogel significantly increased the percentage of adipose tissue in 8-week explants by 1.6-fold and 4-fold in uncrosslinked and 0.03% EDC crosslinked 3% AAM sponges, respectively (p<0.05).

1. A method for producing a tissue product, comprising the steps of:
selecting an adipose tissue;
treating the tissue to remove substantially all cellular material from the tissue;
suspending the tissue in a liquid to form a suspension with a 2-4% by weight solid content; and
freezing and drying the suspension to form a porous sponge.

2. The method of claim 1, further comprising cross-linking the porous sponge.

3. The method of claim 2, wherein crosslinking is performed using a dehydrothermal process.

4. The method of claim 3, further comprising performing a chemical cross-linking step.

5. The method of claim 1, wherein the porous sponge comprises a desired thickness at least in the thickest part of the sponge, the thickness exceeding 10.0 cm.

6. The method of claim 1, further comprising adding the suspension to a mold.

7. The method of claim 6, wherein the mold is in the shape of a round or tear-drop breast implant.

8. The method of claim 4, wherein the chemical cross-linking step includes at least one of glutaraldehyde, genepin, carbodiimides, and diisocyantes.

9. The method of claim 4, wherein cross-linking includes heating the porous sponge.

10. The method of claim 9, wherein the porous sponge is heated in a vacuum.

11. The method of claim 10, wherein the porous sponge is heated to a range of 70° C. to 120° C.

12. The method of claim 4, wherein the porous sponge is cross-linked such that the material maintains the stable three-dimensional structure when contacted with an aqueous environment.

13. The method of claim 12, wherein the aqueous environment is a mammalian body.

14. A tissue product, comprising:
a first component including acellular adipose matrix (AAM); and
a second component including tropoelastin (TE).

15. The tissue product of claim 14, wherein the product is in the form of a sponge.

16. The tissue product of claim 15, wherein the sponge comprises a ratio of AAM to TE of about 3:1 to about 1:3.

17. The tissue product of claim 14, wherein the product is particulate.

18. The tissue product of claim 14, wherein the TE is human TE.

19. The tissue product of claim 14, wherein the AAM is derived from porcine adipose.

20. The tissue product of claim 14, wherein the product has been at least partially cross-linked.

21. The tissue product of claim 20, wherein the cross-linking is performed with 1-ethyl-3-(3-dimethylaminopropyl) carbodiimide hydrochloride (EDC).


SEQUENCE LISTING (26 sequences)

SEQ ID NO: 1 - protein, 8 aa, Unknown
  source: /note=“Description of Unknown tropoelastin peptide”
AAAKAAKM

SEQ ID NO: 2 - protein, 5 aa, Unknown
  source: /note=“Description of Unknown tropoelastin peptide”
GVGVP

SEQ ID NO: 3 - protein, 4 aa, Unknown
  source: /note=“Description of Unknown tropoelastin peptide”
GGVP

SEQ ID NO: 4 - protein, 6 aa, Unknown
  source: /note=“Description of Unknown tropoelastin peptide”
GVGVAP

SEQ ID NO: 5 - protein, 18 aa, Unknown
  source: /note=“Description of Unknown tropoelastin peptide”
GGVPGAIPGGVPGGVFYP

SEQ ID NO: 6 - protein, 9 aa, Unknown
  source: /note=“Description of Unknown tropoelastin peptide”
GVGLPGVYP

SEQ ID NO: 7 - protein, 7 aa, Unknown
  source: /note=“Description of Unknown tropoelastin peptide”
GVPLGYP

SEQ ID NO: 8 - protein, 13 aa, Unknown
  source: /note=“Description of Unknown tropoelastin peptide”
PYTTGKLPYGYGP

SEQ ID NO: 9 - protein, 13 aa, Unknown
  source: /note=“Description of Unknown tropoelastin peptide”
GGVAGAAGKAGYP

SEQ ID NO: 10 - protein, 10 aa, Unknown
  source: /note=“Description of Unknown tropoelastin peptide”
TYGVGAGGFP

SEQ ID NO: 11 - protein, 5 aa, Unknown
  source: /note=“Description of Unknown tropoelastin peptide”
KPLKP

SEQ ID NO: 12 - protein, 12 aa, Unknown
  source: /note=“Description of Unknown tropoelastin peptide”
ADAAAAYKAAKA

SEQ ID NO: 13 - protein, 9 aa, Unknown
  source: /note=“Description of Unknown tropoelastin peptide”
GAGVKPGKV

SEQ ID NO: 14 - protein, 9 aa, Unknown
  source: /note=“Description of Unknown tropoelastin peptide”
GAGVKPGKV

SEQ ID NO: 15 - protein, 9 aa, Unknown
  source: /note=“Description of Unknown tropoelastin peptide”
TGAGVKPKA

SEQ ID NO: 16 - protein, 7 aa, Unknown
  source: /note=“Description of Unknown tropoelastin peptide”
QIKAPKL

SEQ ID NO: 17 - protein, 12 aa, Unknown
  source: /note=“Description of Unknown tropoelastin peptide”
AAAAAAAKAAAK

SEQ ID NO: 18 - protein, 22 aa, Unknown
  source: /note=“Description of Unknown tropoelastin peptide”
AAAAAAAAAAKAAKYGAAAGLV

SEQ ID NO: 19 - protein, 16 aa, Unknown
  source: /note=“Description of Unknown tropoelastin peptide”
EAAAKAAAKAAKYGAR

SEQ ID NO: 20 - protein, 17 aa, Unknown
  source: /note=“Description of Unknown tropoelastin peptide”
EAQAAAAAKAAKYGVGT

SEQ ID NO: 21 - protein, 17 aa, Unknown
  source: /note=“Description of Unknown tropoelastin peptide”
AAAAAKAAAKAAQFGLV

SEQ ID NO: 22 - protein, 29 aa, Unknown
  source: /note=“Description of Unknown tropoelastin peptide”
GGVAAAAKSAAKVAAKAQLRAAAGLGAGI

SEQ ID NO: 23 - protein, 15 aa, Unknown
  source: /note=“Description of Unknown tropoelastin peptide”
GALAAAKAAKYGAAV

SEQ ID NO: 24 - protein, 14 aa, Unknown
  source: /note=“Description of Unknown tropoelastin peptide”
AAAAAAAKAAAKAA

SEQ ID NO: 25 - protein, 12 aa, Unknown
  source: /note=“Description of Unknown tropoelastin peptide”
AAAAKAAKYGAA

SEQ ID NO: 26 - protein, 11 aa, Unknown
  source: /note=“Description of Unknown tropoelastin peptide”
CLGKACGRKRK